(19) United States
(12) Patent Application Publication
(10) Pub. No.: US 2011/0280823 A1
(43) Pub. Date: Nov. 17, 2011

(54) COMPOSITION

(76) Inventor: Timothy John Madden, Wirral (GB)

(21) Appl. No.: 13/127,103

(22) PCT Filed: Sep. 28, 2009
(86) PCT No.: PCT/EP09/62549

§ 371 Date: May 2, 2011

(30) Foreign Application Priority Data
Nov. 7, 2008 (EP) 08168596.8

(51) Int. Cl. A61K 8/46; A61K 8/92; A61Q 5/02

(52) U.S. Cl. 424/70.19; 424/70.24

(57) ABSTRACT

Concentrated shampoo composition comprising from 32 to 38% wt. cleansing surfactant and from 0.5 to 3% wt. coconut oil or palm kernel oil and wherein the composition has a zero shear viscosity of from 2 to 2000 Pa·s and comprises 90% vol. or more composition in the nematic discotic phase.

The present invention relates to a concentrated shampoo.

Despite the prior art there remains the need for improved concentrated shampoo compositions.

EP-A-1 859 777 discloses cosmetic compositions comprising 34.36% wt. surfactant (32.36% wt. Edenor L2SM and 2% wt. Texapon CS) and 8.81% wt. coconut oil, silicone oil and having a viscosity of from 80 to 300 Pa·s.

WO 2005/023208 discloses a shampoo and a liquid soap composition comprising 40% wt. sodium lauryl ethoxy sulphate and 2% wt. coconut oil.

Despite the prior art there remains the need for improved concentrated shampoo compositions.

Accordingly, there is provided a concentrated shampoo composition according to claim 1.

The nematic discotic phase is easily identified by the person of ordinary skill in the art and is characterised by columns of non-axially aligned columns of discs under NMR imaging.

Typically, the phase behaviour for the claimed surfactant system dictates that a regular, non-concentrated shampoo composition will be in the isotropic phase. The hexagonal phase provides a composition which is too viscous to be suitable for the typical consumer and the lamellar phase is not able to deliver actives as well as the isotropic phase so the formulator has little difficulty with phase diagrams when formulating a standard non-concentrated shampoo.

However, as the surfactant concentration is increased the typical composition moves from the isotropic phase to the hexagonal phase.

We have surprisingly found that adding coconut oil or palm kernel oil to compositions which would ordinarily be in the hexagonal phase do not in fact become even thicker, but, instead, become thinner. The resultant compositions are in the nematic discotic phase.

In other words, addition of coconut oil or palm kernel oil to a concentrated composition provides a window of nematic discotic phase which provides similar rheologies to compositions in the isotropic phase.

More surprisingly, compositions can be made without having any significant lamellar structure either. This is what would be expected if one added a significant amount of salt to a composition in the hexagonal phase. Lamellar phase structure is not desired in a shampoo since its ability to deposit materials such as silicones onto the hair is greatly diminished. A composition in the nematic discotic phase is able to deposit materials onto the hair similarly to compositions in the isotropic phase.

Preferably, the zero shear viscosity is from 10 to 200 Pa·s.

Preferably, the composition comprises 95% vol. or more and most preferably 98% or more composition in the nematic discotic phase.

Preferably, the composition comprises from 0.5 to 3% wt. coconut oil or palm kernel oil. More preferably, it comprises from 1 to 2% wt. coconut oil or palm kernel oil.

The cleansing surfactant can be any single or mixture of surfactants commonly used in shampoo compositions. Preferably, the composition comprises surfactant selected from anionic, non-ionic and zwitterionic and mixtures thereof. Most preferably, the cleansing surfactant comprises anionic surfactant.

The level of surfactant present in the concentrated shampoo correlates with the concentrated intent and is typically two or three times higher than that found in regular, unconcentrated shampoos.

Preferably, the cleansing surfactant is present at from 30 to 40% wt.

Preferably, the cleansing surfactant comprises C10-C14 ether sulphate.

In a preferred embodiment the cleansing surfactant comprises sodium lauryl ether sulphate.

In a preferred embodiment the cleansing surfactant comprises sodium lauryl ether sulphate. More preferably, the sodium lauryl ether sulphate comprises an average EO number of from 1 to 3 and is most preferably from 1 to 1.4.

The concentrated shampoo may also comprise a co-surfactant. Preferred co-surfactants are the amphoteric surfactants. More preferably, the co-surfactant is cocoamidopropyl betaine or cocamide MEA.

Preferably, the composition comprises C10-C14 ether sulphate and cocoamidopropyl betaine or cocamide MEA in a ratio of from 11:1 to 17:1, more preferably from 13:1 to 15:1.

Preferably, the concentrated shampoo comprises an oil. The change in phase structure effected by the coconut oil or palm kernel oil enables the concentrated shampoo composition to carry materials that would not be possible outside the nematic discotic phase. Suitable materials include oils.

Preferably, the oil is selected from mineral oil, vegetable oil, animal oil, or mixtures thereof and more preferably mineral oil.

### EXAMPLE 1

Coconut Oil

| | | | | | | | | | |
|---|---|---|---|---|---|---|---|---|---|
| % SLES | 14 | 21 | 24.5 | 28 | 31.5 | 35 | 38.5 | 42 | 49 |
| % CAPB | 1 | 1.5 | 1.75 | 2 | 2.25 | 2.5 | 2.75 | 3 | 3.5 |
| total | 15 | 22.5 | 26.25 | 30 | 33.25 | 37.5 | 41.25 | 45 | 52.5 |
| E | | | L1/L2 | L1/L2 | ND/L2 | ND/L2 | La | La | La |
| D | L1/L2 | L1/L2 | L1/L2 | L1 | ND/L2 | ND | La | La | La |
| C | L1/L2 | L1/L2 | L1 | L1 | ND | ND | La | La | La |
| B | L1/L2 | L1/L2 | L1 | L1/ND | ND | ND | H1/ND | H1/La | La |
| A | L1 | L1 | L1/H1 | L1/H1 | H1 | H1 | H1 | H1 | La |

A 0% Coconut Oil
B 1% Coconut Oil
C 2% Coconut Oil
D 3% Coconut Oil
E 4% Coconut Oil

Where L1 is isotropic phase; L2 is isotropic oil; H1 is hexagonal phase; ND is the nematic discotic phase and La is the lamellar phase.

### EXAMPLE 2

| | | | % Active |
|---|---|---|---|
| | % Active | % wt. | in material |
| Sodium Laureth Sulphate | 35 | 50 | 70% |
| Cocamide MEA | 2.5 | 2.5 | 100% |
| Dimethiconol/TEA-DOBS | 5 | 10 | 50% |
| Coconut Oil | 2 | 2 | 100% |
| Parfum | 1 | 1 | 100% |
| Guar Hydroxypropyl Trimonium | 0.2 | 0.2 | 100% |
| Chloride | | | |
| DMDM Hydantoin and 3-iodo- | 0.2 | 0.4 | 50% |
| 2propylnylbutyl carbamate | | | |
| Aqua | q.s. to 100 | q.s. to 100 | 100% |

### EXAMPLE 3

### Method for Preparation of Small Samples, Ascertaining Ratio of Multiple Phase if Present and Identifying Phase Structures

Formulations were prepared in 5 g batches, all the ingredients of the test formulation were added to a 20 ml crimp top glass GC vial, stoppered with butyl rubber bung and crimped closed with aluminium crimp. They were then heated for 3-4 hours or overnight in an oven at 85° C. The samples were allowed to cool, unsealed, mixed, resealed and heated again to 85° C. for 3-4 hours or overnight. This cycle was repeated until the sample was homogeneous or there was no change on reheating. Once cooled the samples in vials were visually assessed to identify if the samples had separated into two or more layers indicating multiple phases present. The ratio of the amounts of the phases present can be obtained by measuring the vertical distance each phase occupies in the vial. The cooled samples were examined by polarising optical microscopy, Olympus BX51 with 10× objective, using the observed textures to identify the resulting surfactant phases (S. Hassan, W. Rowe & G J T. Tiddy, Handbook of Applied Surfactant and Colloid Chemistry Vol. 1, p 465). If multiple phases were present a sample of material from each phase was extracted and examined individually.

### Method for Measuring Zero Shear Viscosity

Rheology of shampoos were measured with a TA Instruments ARG2 Control Stress Rheometer at 25° C. The zero shear viscosity value was taken to be the measured shear viscosity at the shampoo's yield stress. The yield stress was identified from a strain frequency sweep.

1. Concentrated shampoo composition comprising from 32 to 38% wt. cleansing surfactant and from 0.5 to 3% wt. coconut oil or palm kernel oil and wherein the composition has a zero shear viscosity of from 2 to 2000 Pa·s and comprises 90% vol. or more composition in the nematic discotic phase, wherein the cleansing surfactant comprises C10-C14 ether sulphate.

2. Composition according to claim 1 wherein the zero shear viscosity is from 10 to 200 Pa·s.

3. Composition according to claim 1 comprising 95% vol. or more composition in the nematic discotic phase.

4. Composition according to claim 1 comprising a further oil.

5. Composition according to claim 4 wherein the further oil is selected from mineral oil, vegetable oil, animal oil, or mixtures thereof.

6. Composition according to claim 1 comprising cocoamidopropylbetaine or cocamide MEA.

7. Composition according to claim 6 wherein the cleansing surfactant comprises C10-C14 ether sulphate and cocoamidopropylbetaine or cocamide MEA in a ratio of from 11:1 to 17:1, more preferably from 13:1 to 15:1.

8. Composition according to claim 7 wherein the ratio of C10-C14 ether sulphate to cocoamidopropylbetaine or cocamide MEA is 13:1 to 15:1
